(19) United States
(12) Patent Application Publication
(10) Pub. No.: US 2012/0315662 A1
(43) Pub. Date: Dec. 13, 2012

(54) METHOD FOR THE EARLY DETECTION OF HIGH-GRADE PELVIC SEROUS CANCER

(76) Inventor: Georgiann C. Linnemeier, Carmel, IN (US)

(21) Appl. No.: 13/488,469

(22) Filed: Jun. 5, 2012

Related U.S. Application Data
(60) Provisional application No. 61/520,278, filed on Jun. 7, 2011.

(51) Int. Cl. A61B 10/02; C12Q 1/02

(52) U.S. Cl. 435/29; 600/569

(57) ABSTRACT

A method for early detection of high-grade pelvic serous cancers, comprising acquiring fallopian tube cells in vivo by exfoliative cytology, and examining the acquired cells for precursors of high-grade pelvic serous cancer.

### RELATED APPLICATIONS

This application claims a right of priority under 35 U.S.C. §119 upon U.S. Provisional Patent Application Ser. No. 61/520,278, that was filed Jun. 7, 2011, and which is incorporated by reference herein.

### FIELD OF THE INVENTION

The present invention relates generally to methods for the early detection of cancers, and more particularly to a minimally invasive method for the early detection of high-grade pelvic serous cancers.

### BACKGROUND OF THE INVENTION

One of the greatest accomplishments in gynecologic cancer prevention in the past 50 years has been the widespread use of the Papanicolaou smear (Pap test). This success is the result of the detection of a continuum of pre-invasive lesions of the cervix that are detected far more frequently than is invasive cervical cancer. The past and the future success of this technique has depended heavily on two characteristics of cervical carcinogenesis: first, these tumors are preceded by a precursor lesion that remains non-invasive for as long as 20 years, and second, these changes are linked to the human papilloma virus (HPV), for which a vaccine is now available.

The Pap test is the most widely used cervical cancer screening method. It is a simple procedure in which a small sample of cells is collected with a brush from the cervix and examined microscopically.

Increasing knowledge about the etiology of cervical cancer, combined with the development of molecular diagnostic technology, has introduced a new level of specificity to cervical cancer screening. Moving diagnostic testing from the cellular level to the molecular level has allowed not only the identification of the existing precancerous state, but also has refined the spectrum of the disease.

Discovering critical cervical biomarkers to clarify the risk of progression in the pathogenesis of cervical cancer using an exfoliative specimen has been studied. Analyzing epidemiologic, pathologic and molecular biologic alterations in women with the full spectrum of HPV infections, pre-cancer, and cancer has led to the development of categories of risk and categories which predict progression and invasion. See, Wentzensen, N., et al., Grading the severity of cervical neoplasia based on combined histopathology, cytopathology, and HPV genotype distribution among 1,700 women referred to colposcopy in Oklahoma. Int J Cancer, 2009. 124(4): p. 964-9, and Hu, X., et al., A microRNA expression signature for cervical cancer prognosis. Cancer Res. 70(4): p. 1441-8.

A majority of patients with signs and symptoms of malignant biliary obstruction are ultimately not suitable for surgery, either due to locally invasive or metastatic disease, or because of underlying medical problems. Only 10-15% of pancreatic cancers and less than half of colangiocarcinomas are resectable. For inoperable patients, therapy with either chemotherapy or radiotherapy requires a definitive pathologic diagnosis. In addition, the quality of information provided to the patient regarding prognosis is severely compromised if the exact diagnosis is unknown. Endoscopic retrograde colangiopancreatography (ERCP) with biliary brush cytology has been the initial investigation of choice for cytopathological diagnosis in jaundiced patients with suspected pancreaticobiliary malignancy. See the Cytomax II® Double Lumen Biliary Cytology Brush of Cook Medical, Inc., Bloomington, Ind. Although this technique has a specificity of 100%, sensitivity for malignancy has been reported as ranging from 15-80%. For this reason, molecular markers are being evaluated for their potential utility to aid in the diagnosis of pancreaticobiliary lesions.

Molecular markers such as p53 have been studied. The diagnostic yield of p53 immunocytology for the detection of malignancies in material obtained by biliopancreatic tree brushing detected 87% of the malignancies in one study group. See 3 Villanacci, V., et al., Immunocytochemical assessment of p53 protein to detect malignancy in increased cell-yield brush cytology from the biliopancreatic tree. Dig Dis Sci, 2009. 54(4): p. 789-92.

Another study demonstrated a sensitivity of 88.2% when cytology was combined with p53 immunocytology. See Kim, Y. S., et al., The Significance of p53 and K-ras Immunocytochemical Staining in the Diagnosis of Malignant Biliary Obstruction by Brush Cytology during ERCP. Gut Liver. 4(2): p. 219-25. Recently, a gene panel was identified that was able to differentiate patients with benign versus malignant endoscopic brush samples with high accuracy. The authors suggested that ultimately, if molecular markers such as aberrant genes can be used to help diagnose periampullary cancer, these markers could also potentially be useful in identifying microscopic preinvasive neoplastic disease (PanINs). Detecting PanINs is particularly important for patients with an inherited predisposition to pancreatic and other periampullary cancers. Resected pancreata of many high-risk individuals have been shown to harbor preinvasive neoplastic disease. The ability to reliably detect and quantify PanIN using molecular assays in high-risk individuals would help identify individuals needing more surveillance to detect advanced pancreatic neoplasia. See Parsi, M. A., et al., DNA methylation alterations in endoscopic retrograde cholangiopancreatography brush samples of patients with suspected pancreaticobiliary disease. Clin Gastroenterol Hepatol, 2008. 6(11): p. 1270-8.

Worldwide every year 190,000 women develop ovarian cancer and more than 140,000 die from the disease. Ovarian cancer occurs when a cell on the surface of the ovary or in the fallopian tubes acquires genetic changes that allow it to grow uncontrollably and to spread around the body. For women whose ovarian cancer is diagnosed when it is confined to the site of origin (Stage I) the outlook is good; 70-80% of these women survive for at least 5 years. However, very few ovarian cancers are diagnosed this early. Usually by the time the ovarian cancer causes symptoms, it has spread into the pelvis (Stage II disease); into the space around the bowels, stomach and liver (Stage III disease); or to distant organs (Stage IV disease). Patients with advanced-stage ovarian cancer are treated with surgery and chemotherapy, but despite recent treatment improvements, only 15% of women diagnosed with Stage 1V disease survive for 5 years.

Most deaths from ovarian cancer are caused by serous ovarian cancer, a tumor subtype that is rarely diagnosed before it has spread. High-grade serous ovarian cancer is therefore a lethal disease for which improved screening and treatment strategies are urgently needed. Early screening, detection and treatment of serous ovarian cancer would save the lives of many women, but until recently, no one knew what these cancers look like before they spread or how long they have grown before they become clinically apparent. Recent findings suggest that the time period over which the early detection of serous ovarian cancer would save lives is surprisingly long. A test that is sensitive and specific enough to take advantage of this “window of opportunity” is needed, yet progress in these areas is impeded by our poor understanding of how this cancer develops.

Ovarian neoplasms consist of several histopathological entities and treatment depends on the specific tumor type. Epithelial ovarian cancer comprises the majority of malignant ovarian neoplasms (about 80%) with the high-grade serous subtype being the most common within this group. In the United States, ovarian cancer accounts for 5% of all cancer deaths in women. In the year 2010, there were an estimated 13,850 deaths due to ovarian cancer. Less than 40% of women with ovarian cancer are cured. The incidence of ovarian cancer increases with age and is most prevalent in the eighth decade of life, with a rate of 57/100,000 women. The median age at the time of diagnosis is 63 years, and 70% of patients have advanced disease.

Epidemiologic studies have identified risk factors in the etiology of ovarian cancer. A 30% to 60% decreased risk of cancer is associated with younger age pregnancies, with first births at 25 years of age or younger, with the use of oral contraceptives, and/or with breast feeding. Conversely, nulliparity or being older than 35 at first birth confers an increased risk of cancer. In addition, a recent meta-analysis of 21 studies confirmed a protective effect of tubal ligation on the risk of invasive ovarian cancer.[1, 2] A family history of ovarian cancer, including BRCA1 and BRCA2 genotypes, or families with Lynch II syndrome, has also been found to be associated with the early-onset of disease. Environmental factors have also been investigated, but so far they have not been conclusively associated with this neoplasm.

Early ovarian cancer usually presents no obvious symptoms. There is currently no sufficiently accurate screening test that has been proven to be effective in the early detection of ovarian cancer. Trials to assess multimodality screening with ultrasound and cancer antigen 125 (CA-125) have demonstrated no increased detection of early stage cancer; in fact, 72% of the cancers detected were late stage. See Partridge, E., et al., Results from four rounds of ovarian cancer screening in a randomized trial. Obstet Gynecol, 2009. 113(4): p. 775-82.

Novel screening methods are urgently needed for all women, especially those with a genetic predisposition to high-grade pelvic serous cancer. Early detection will save lives.

### SUMMARY OF THE INVENTION

The method of fallopian tube cell acquisition for the early diagnosis of pelvic serous cancer of the present invention (hereinafter “FAP”) is a novel method that is designed to sample cells of the fallopian tubes in vivo in order to detect cellular changes and other precursors early in the natural history of serous ovarian cancer. Recent studies have revealed that the fallopian tubes can display areas of increased, abnormal cellular growth that are precursors of cancer. Identifying these pre-cancerous changes early in their development would enable early surgical interventions and result in significantly higher cure rates for high-grade pelvic serous cancer.

One preferred embodiment of the present invention is a method for early detection of high-grade pelvic serous cancers, comprising acquiring fallopian tube cells in vivo by exfoliative cytology, and examining the acquired cells for precursors of high-grade pelvic serous cancer.

Another preferred embodiment of the present invention is a method for early detection of high-grade pelvic serous cancers, comprising acquiring fallopian tube cells in vivo by exfoliative cytology wherein the cells are acquired by a sheathed cytology brush, and examining the acquired cells for precursors of high-grade pelvic serous cancer.

Another preferred embodiment of the present invention is a tool for acquiring fallopian tube cells in vivo by exfoliative cytology, comprising, a sheathed cytology brush having an outer diameter of 5F and a length of 50 cm, with a nylon-bristled cytology brush having a diameter of 3 mm and a length of 1 cm, and with a radiopaque distal end cap, and with a sheathed brush hub having a side arm connector with a Luer lock and a ringed handle with an excursion of 5-7 cm that allows the cytology brush to extend 5-7 cm out of the distal tip of the sheath when the ring is fully pushed into the hub.

### BRIEF DESCRIPTION OF THE DRAWINGS

FIG. 1 is an illustration of a fallopian tube.

FIG. 2 is an illustration of a cross section of a fallopian tube.

### DETAILED DESCRIPTION OF A PREFERRED EMBODIMENT

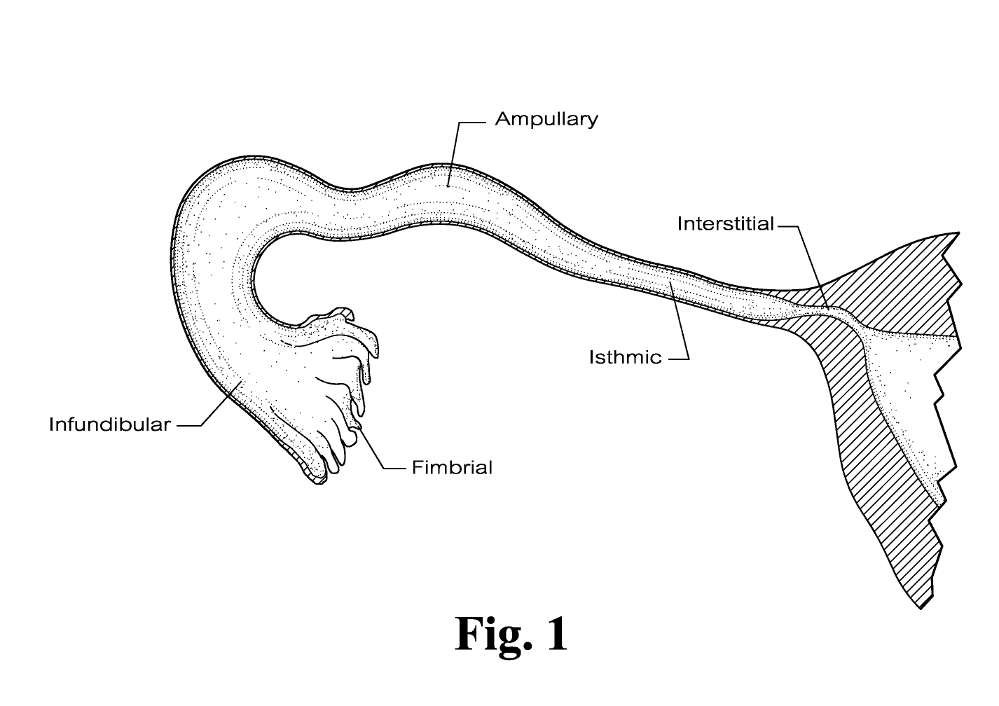

Most ovarian cancer research is based on the hypothesis that ovarian cancer arises from the ovarian surface epithelial cells. However, a recent series of paradigm-shifting studies has suggested that over 50% of high-grade serous carcinomas involving the ovary likely arise from the fallopian tubes. See e.g. Kurman, R. J. and M. Shih Ie, The origin and pathogenesis of epithelial ovarian cancer: a proposed unifying theory. Am J Surg Pathol. 34(3): p. 433-43. Referring to FIG. 1, the fallopian tubes are paired, tubular, seromuscular organs that are the anatomic pathway to human reproduction. Each tube is about 10 cm (range, 7-14 cm) in length. It is an elongated trumpet-shaped structure that extends from the cornu of the uterine cavity to the ovary. Its fimbriated end, which is open to the peritoneal cavity, sweeps over the ovary, allowing the ovulated egg to be pulled into the fallopian tube where fertilization occurs. The fimbriae, the infundibulum, and the thin-walled ampulla form more than half of the length of the fallopian tube; passing medially, the isthmic portion and the intrauterine portion comprise the remainder of the tube. The intrauterine portion of the fallopian tube is about 1 cm in length and 1 mm in inner diameter.

Fallopian tube epithelium can exhibit areas of increased proliferation and cytologic atypia, called intraepithelial neoplasia. Serous ovarian carcinomas frequently exhibit mutations in the critical cell cycle regulator p53. Identical p53 mutations have been identified in tubal intraepithelial carcinoma (TIC) with coexisting sporadic serous carcinoma, suggesting that genetic disruption within the fallopian tube may progress to ovarian carcinoma. The fact that scientists have been looking for early cancer in the wrong anatomical location may explain why so few high-grade serous ovarian tumors have been detected at an early stage. Hence, the term high-grade pelvic serous carcinoma (HGPSC) would seem to be more accurate.

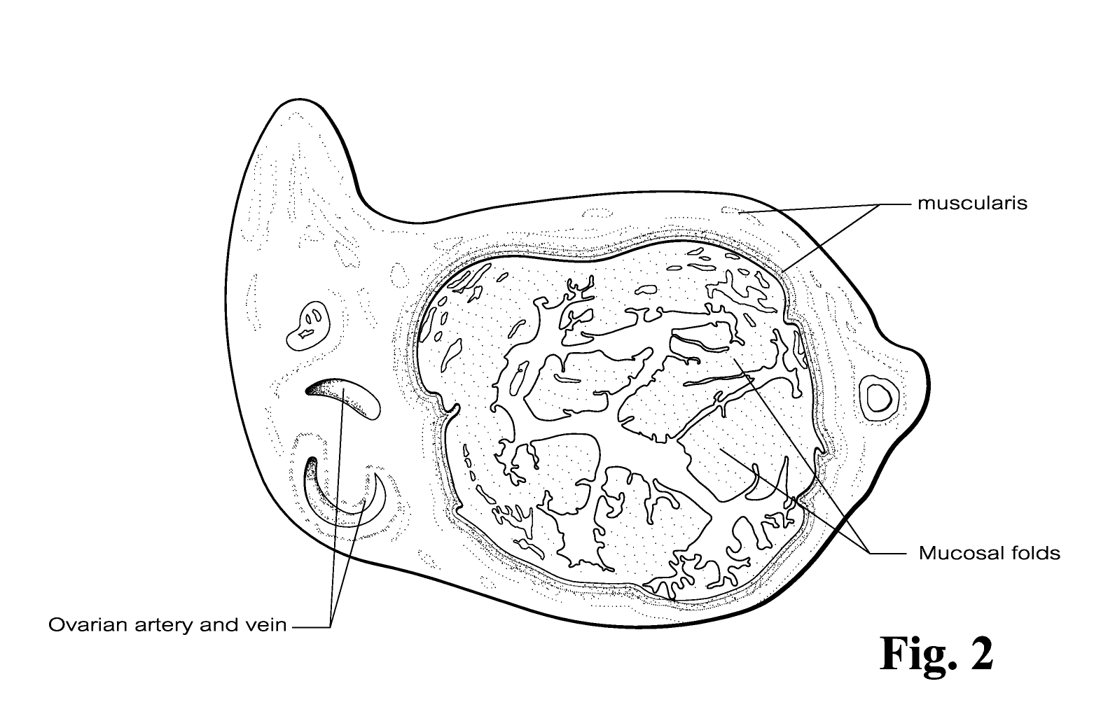

Each fallopian tube is formed from the Mullerian system and lies within the broad ligament located between the ovary and the uterus. It is divided into the infundibulum that opens onto the peritoneal cavity and is ringed by fimbrial projections, the more proximal ampulla, and the isthmus, which merges with the uterus. Referring now to FIG. 2, the mucosa consists of a nonstratified epithelial lining composed of three cell types, ciliated, secretory, and intercalated cells. The most common are the ciliated cells, followed by the secretory cells, which together comprise over 90% of the cell population. The intercalated cells are presumed to be a variant of the secretory cells. Variations in the cellular makeup of the tubal mucosa depend upon reproductive age, hormonal status, and location within the tube. Recent studies have indicated that preneoplastic changes arise in the secretory cells and that relevant genetic alterations can transform these cells into high-grade serous carcinoma. See Karst, A. M., K. Levanon, and R. Drapkin, Modeling high-grade serous ovarian carcinogenesis from the fallopian tube. Proc Natl Acad Sci USA. 108(18): p. 7547-52; and Karst, A. M. and R. Drapkin, Ovarian cancer pathogenesis: a model in evolution. J. Oncol. 2010: p. 932371.

Significant progress has been made in the development of fallopian tube epithelium-based HSPSC models. A primary cell culture system in which human epithelial cells are isolated from a fresh sample of normal fallopian tube has been used to reconstruct an intact fallopian tube epithelium outside the body. See Levanon, K., et al., Primary ex vivo cultures of human fallopian tube epithelium as a model for serous ovarian carcinogenesis. Oncogene. 29(8): p. 1103-13.

This model has been used to examine cellular responses to environmental stress, mechanical damage, DNA damage, cytokines, or inflammatory elements. This model has shown that fallopian tube epithelial secretory cells exhibit delayed DNA damage response kinetics compared to neighboring ciliated cells following genotoxic insult.

Further insight into the development of HGPSC has been gained utilizing transformed human fallopian tube secretory cells. Systematic evaluation of fallopian tube secretory cells with individual gene alterations, when xenografted into mice, gave rise to disseminated tumors that metastasized throughout the peritoneal cavity. Histologic immunophenotyping and serum biomarkers validated that these tumors were high-grade serous carcinomas. See Levanon, K., et al., supra. Further evidence for a tubal origin is suggested by the high prevalence of occult fallopian tube carcinomas identified among BRCA1 and BRCA2 mutation carriers undergoing risk-reducing salpingo-oophorectomy (RRSO). Although the lifetime risk of ovarian carcinoma in the general population is only 1% to 2%, women who inherit mutations in the BRCA1 and BRCA2 genes have up to a 50% lifetime risk of ovarian carcinoma. These high-risk women are frequently discovered to have occult neoplasms at the time of RRSO, and 57% to 100% of these lesions arise in the fallopian tubes. Fallopian tube epithelium frequently contains areas that have been termed p53 foci (also referred to as p53 signatures), which overexpress p53 and have increased expression of the proliferation marker Ki-67. These tubal p53 foci are more frequent in fallopian tubes from BRCA1 and BRCA2 mutation carriers compared with normal-risk women, and they have also been shown to exhibit decreased expression of the tumor suppressor protein p27. See Press, J. Z., et al., Identification of a preneoplastic gene expression profile in tubal epithelium of BRCA1 mutation carriers. Neoplasia. 12(12): p. 993-1002.

These observations have resulted in the proposal of a new paradigm for ovarian carcinoma, in which the fallopian tube epithelium acquires a sequence of molecular abnormalities leading to an in situ or invasive neoplasm, which exfoliates and spreads to the ovary and peritoneum. Identification of these precursor lesions has thus far been by pathology ex vivo after surgical removal of the fallopian tubes and ovaries. Modeling for the growth and progression of these precancerous lesions suggests that there may be a window of detection encompassing several years. See Brown, P. O. and C. Palmer, The preclinical natural history of serous ovarian cancer: defining the target for early detection. PLoS Med, 2009. 6(7): p. e1000114.

The equipment and technique for transcervical fallopian tube catheterization are extensions of hysteroscopy. The hysteroscope provides a sterile conduit through which a series of coaxial catheters and guidewires can be introduced. There are several basic steps to the procedure: (1) introduction of the hysteroscope into the cervical os (this may require dilation of the os), (2) achieving uterine distention, (3) achieving uterine cornual visualization, (4) achieving fallopian tube ostial visualization (both left and right), and (5) advancing the catheter into the fallopian tube.

Use of selective salpingography and fallopian tube recanalization has contributed to the diagnosis and treatment of infertility. Transcervical cannulation of the proximal fallopian tube has been demonstrated to be an effective method for evaluating and treating obstruction. See Novy, M. J., et al., Diagnosis of cornual obstruction by transcervical fallopian tube cannulation. Fertil Steril, 1988. 50(3): p. 434-40.

The Novy Cornual Cannulation Set of Cook Medical, Inc., Bloomington, Ind., is used for hysteroscopic selective catheterization of the proximal fallopian tube. The catheter set is introduced and utilized through the operating channel of a hysteroscope. The introducing catheter (5F) is available curved or straight, the length is 35 cm, the inner catheter is 3F with a catheter length of 50 cm. The guide wire diameter is 0.018 inch.

In a similar manner, contraception may be achieved by placing permanent inserts in the fallopian tubes. The Essure and Adiana devices are two contraception examples.

Essure: Using a transcervical approach, one Essure micro-insert is placed in the proximal portion of each fallopian tube lumen. The micro-insert is a dynamically expanding micro-coil 4 cm in length and 0.8 mm in diameter in its wound down configuration. When released from the delivery system, the outer coil expands to 1.5 to 2.0 mm in diameter. The Essure micro-insert is provided attached to the delivery wire in a wound-down configuration. The delivery wire is composed of a nitinol core wire which is ground at the distal end to result in a flexible, tapered profile. The device is constrained and sheathed in a flexible delivery catheter. A black positioning marker on the delivery catheter aids in proper placement of the device in the fallopian tube.

Adiana: A catheter used to apply radiofrequency (RF) energy is introduced into the intramural section of the fallopian tube through a conventional hysteroscope, via a transvaginal and transcervical approach. Confirmation of correct catheter positioning within the intramural tube is achieved by means of direct visual assessment through the hysteroscope to confirm that the black positioning mark on the catheter has reached the tubal ostium. Once the RF generator has signaled that the catheter is correctly positioned, the clinician activates the generator. The RF generator delivers bipolar RF energy (<3 Watts) that creates a superficial lesion within the fallopian tube. The clinician then depresses the matrix release button on the catheter to deploy the silicone matrix within the region of the lesion. The outer sheath retracts while the push rod keeps the matrix in place. The catheter is then removed and the procedure repeated with a new catheter on the contralateral fallopian tube.

Difficulty in advancing the catheter into a cornu, or difficulty advancing the catheter in the fallopian tube can arise from spasm, normal anatomic variation, or acquired uterine or tubal abnormalities. Overall, experienced operators report a 71-92% success in recanalization of the fallopian tube.

Possible adverse effects have included:
1. Damage to normal fallopian tubes: It is unknown whether this procedure may damage normal fallopian tubes or put the patient who has normal tubes at any increased risk for subsequent ectopic pregnancy.
2. Tubal dissection: It is possible that the guide wire, catheter or brush may dissect into the wall of the tube and cause blockage or narrowing of the lumen. This might result in sterility or infertility or increased risk of ectopic pregnancy.
3. Pain or discomfort: Patients may experience mild cramping from distention of the uterus or instrumentation of the fallopian tube.
4. Tubal perforation, bleeding Infection: The fallopian tube is relatively thick and muscular and not easily punctured; however, tubal perforation has been reported in 2-4% of patients and is usually related to the severity of underlying tubal disease. Bleeding or infection can occur. Patients may experience light spotting of blood after the procedure mainly due to cervical or endocervical manipulation. The exclusive use of sterile instruments minimizes the risk of infection.

The novel FAP method of the present invention employs techniques similar to those currently used for the promotion and prevention of pregnancy that utilize relatively simple and inexpensive tools to provide a low-risk, minimally invasive approach to the early diagnosis of pelvic serous cancer by acquiring cells from the fallopian tubes for cytologic and molecular analysis. These promotion and prevention of pregnancy procedures come with no major safety concerns and a reported technical success rate of 70-88%.

Two categories of methods are involved in obtaining cells for microscopic examination. The first is to obtain the medium that contains naturally exfoliated cells. The second, which is used in the novel FAP method of the present invention, is to specifically obtain the cells of interest for examination with an instrument, such as a brush. The preparation of cytologic specimens when the cells are collected with an instrument, requires the cells to be either rinsed into a preservative solution, or simply spread directly onto slides. When the cells are collected in a medium, whether natural or artificial, the specimen needs to go through a process in order to separate the medium from the cells. This can be done with centrifugation or filtering. The optimal final product for every type of preparation is a slide with a thin layer of evenly dispersed cells on it. Cells thinly spread on a slide dry out very easily; therefore, the slides need to be fixed in 95% ethanol immediately and then stained with Papanicolaou or hematoxylin and eosin stain. An experienced cytopathologist then examines the prepared slide for cytologic abnormalities.

Specific benefits of cytologic analysis include cost-effectiveness, rapid turnaround time, and a diagnosis with minimal patient risk. Since cytologic specimens usually consist of a very small amount of cells or tissue fragments, an optimal technique for both sample collection and slide preparation is crucial. Such molecular analyses for the diagnosis of cancer by utilizing specimens obtained by brush cytology is being studied in the oral cavity [see Liu, X., et al., MicroRNA-24 targeting RNA-binding protein DND1 in tongue squamous cell carcinoma. FEBS Lett. 584(18): p. 4115-20; Perez-Sayans, M., et al., Measurement of ATP6V1C1 expression in brush cytology samples as a diagnostic and prognostic marker in oral squamous cell carcinoma. Cancer Biol Ther. 9(12): p. 1057-64; and Weigum, S. E., et al., Nano-bio-chip sensor platform for examination of oral exfoliative cytology. Cancer Prey Res (Phila). 3(4): p. 518-28] and in the esophagus [see Huang, Q. and L. J. Hardie, Biomarkers in Barrett's oesophagus. Biochem Soc Trans. 38(2): p. 343-7].

The novel FAP method of the present invention will allow the utilization of cytologic and molecular techniques similar to those currently being employed with great success in other anatomic sites, such as the cervix, the pancreaticobiliary tree, the oral cavity, and the esophagus.

The Indications for Use of the Method of the Present Invention are:

The novel FAP procedure of the present invention is indicated for women who are at risk for developing high-grade pelvic serous cancer and who desire further diagnostic evaluation.

The Contraindications are:

Patients who have previously undergone tubal surgery or placement of devices within the fallopian tubes;

Patients with known reproductive tract anatomical variants such as bicornuate uterus or leiomyoma that would preclude visualization of the tubal ostia;

Pregnancy or suspected pregnancy; Active or recent upper or lower pelvic infection;

Delivery or termination of a pregnancy less than 6 weeks previous; and

Known or highly suspected diagnosis of pelvic serous cancer.

The Benefits are:

It does not require incisions; and

It can be performed with or without general anesthesia

### An Exemplary FAP Procedure

Pre-Procedure Testing:

Prior to undergoing the novel FAP procedure of the present invention, patients should undergo a pelvic exam to rule out active infection and to assist in determining the position of the uterus for easier introduction of the hysteroscope.

Facility Requirements:

The novel FAP procedure of the present invention can be performed in an inpatient, outpatient or an office surgery setting.

Appropriate equipment, medications, staff and training should be in place to handle emergency situations such as vaso-vagal response.

Sterile techniques should always be used during the procedure following universal precautions.

Face and eye protection should be worn during the procedure.

The procedure should not exceed 20 minutes (10 minutes per fallopian tube).

The procedure should be supported by an assistant to handle all sterile instruments, and to obtain and provide supplies.

Scheduling:

The novel FAP procedure of the present invention should be performed during the early proliferative phase of the menstrual cycle to enhance the visualization of the tubal ostia.

A pregnancy test should be conducted prior to the procedure.

Pain/Discomfort Expectations:

The patient should be reminded that she may experience pain/discomfort during the procedure.

Pre-Procedure Medications (as Applicable):

A non-steroidal anti-inflammatory drug is recommended one to two hours before the procedure.

An anxiolytic agent may be given 30 minutes prior to the procedure.

The Fallopian Cytology Brush:

The sheathed cytology brush will be of 5F outer diameter and 50 cm in length to fit in the standard operating channel of a hysteroscope.

The cytology brush will be flexible and have nylon bristles with a brush diameter of 3 mm and a brush length of 1 cm, and the brush wire will be made of radiopaque twisted stainlees steel wire with a radiopaque distal end cap.

The sheathed brush hub will have a side arm connector with a Luer lock.

The sheathed brush hub will have a ringed handle with an excursion of 5-7 cm, which will allow the brush to extend 5-7 cm out of the distal tip of the sheath when the ring is fully pushed into the hub.

All components will be sterile, disposable, and intended for a one-time use with easy access to the essential tools in one convenient kit.

The Novel FAP Procedure:

Place the patient in the lithotomy position and drape her per standard procedure. Introduce a bi-valve, open-sided speculum into the vagina to allow access to the cervix, and prep the cervix with antibacterial solution.

Administer a local anesthetic (paracervical block) (as applicable).

Connect a camera, light source, sealing cap, fluid inflow and outflow tubing to the hysteroscope.

Insert the sheathed cytology brush through the sealing cap on the hysteroscope working channel and guide it through until the distal tip of the catheter emerges from the opening of the distal tip of the hysteroscope.

Open the inflow port and close the outflow port on the hysteroscope.

Flush the scope of all air bubbles.

Focus the hysteroscope, perform a white balance and check the inflow/outflow functions.

While the irrigation is on, insert the sterile hysteroscope, with a sheathed cytology brush in place, through the cervix into the uterine cavity.

Cervical dilation may be necessary to allow hysteroscope insertion.

Remove the speculum.

Uterine cavity distention should be accomplished with pre-warmed 0.9% normal saline infusion introduced under gravity feed through the inflow channel of the hysteroscope (infusion under pressure may be needed for adequate uterine distention).

Standard fluid monitoring procedures should be followed throughout the procedure.

Once the tubal ostium is visualized, direct the sheathed cytology brush towards the tubal orifice and place the tip of the sheath at the tubal ostium.

Remove the Luer lock cap from the side arm of the sheathed brush hub and attach a 3 ml syringe.

While maintaining suction with the syringe, advance the cytology brush and make 6-8 passes with the brush and then retract the brush into the sheath.

Remove the sheath and cytology brush from the hysteroscope.

Expel any fluid collected in the sheath or syringe in a 2 cc eppendor tube.

Smear the brush on a glass slide and place the slide immediately in 95% alcohol.

Place the brush in the eppendor tube with a quantity sufficient to total 1.5 ml normal saline and agitate to remove cells.

The specimen will be centrifuged for four minutes in a microcentrifuge, the supernate decanted and the pellet stored at −20 deg C.

Slides will be examined by an experienced cytopathologist.

Cell pellets will be sent for further molecular analysis.

Repeat the novel FAP procedure on the contralateral fallopian tube.

Remove the hysteroscope.

Conclude the novel FAP procedure.

An estimated 13,850 deaths due to ovarian cancer occurred in 2010. Ovarian cancer causes more deaths each year than any other cancer of the female reproductive system. Early ovarian cancer usually has no obvious symptoms. There is currently no sufficiently accurate screening test proven to be effective in the early detection of ovarian cancer. If diagnosed at the localized stage, the 5-year survival rate is 94%; however, only 15% of all cases are detected at this stage. The majority of cases (62%) are diagnosed at an advanced stage with 5-year survival rates of 28%.

The novel FAP procedure of the present invention is designed to detect precancerous changes in the fallopian tubes before these changes progress to invasive cancer. Specific benefits include cost-effectiveness and an early definitive diagnosis with minimal patient risk.

It will be understood that this disclosure is only illustrative. Changes may be made in details, particularly in matters of size, material, and arrangement of parts of the preferred cytology brush without exceeding the scope of the invention. Additionally, the method of the present invention has been described as diagnostic in scope; however, other applications such as providing prognostic and therapeutic information are also envisioned.

What is claimed is:

1. A method for early detection of high-grade pelvic serous cancers, comprising
acquiring fallopian tube cells in vivo by exfoliative cytology, and examining the acquired cells for precursors of high-grade pelvic serous cancer.

2. A method for early detection of high-grade pelvic serous cancers, comprising
acquiring fallopian tube cells in vivo by exfoliative cytology wherein the cells are acquired by a sheathed cytology brush, and
examining the acquired cells for precursors of high-grade pelvic serous cancer.

3. The method of claim 2 wherein the sheathed cytology brush of the acquiring step has an outer diameter of 5F and a length of 50 cm, and a flexible radiopaque nylon-bristled cytology brush with a diameter of 3 mm and a length of 1 cm, with a radiopaque distal end cap, and a sheathed brush hub with a side arm connector with a Luer lock and with a ringed handle with an excursion of 5-7 cm that allows the cytology brush to extend 5-7 cm out of the distal tip of the sheath when the ring is fully pushed into the hub.

4. A tool for acquiring fallopian tube cells in vivo by exfoliative cytology, comprising, a sheathed cytology brush having an outer diameter of 5F and a length of 50 cm, with a flexible radiopaque nylon-bristled cytology brush with a diameter of 3 mm and a length of 1 cm, and with a radiopaque distal end cap, and a sheathed brush hub having a side arm connector with a Luer lock and a ringed handle with an excursion of 5-7 cm that allows the cytology brush to extend 5-7 cm out of the distal tip of the sheath when the ring is fully pushed into the hub.
